(19) United States
(12) Patent Application Publication
(10) Pub. No.: US 2026/0115647 A1
(43) Pub. Date: Apr. 30, 2026

(54) CARBON FOOTPRINT REDUCTION FOR IMPURITIES REMOVAL FROM A PRODUCED GAS

(71) Applicant: ChampionX LLC, Sugar Land, TX (US)

(72) Inventors: Alan Maxwell Blair, Southampton (GB); Keith Norman Garside, Cadnam (GB); Jeffery Caleb Clark, Sugar Land, TX (US)

(21) Appl. No.: 18/990,102

(22) Filed: Dec. 20, 2024

Related U.S. Application Data
(60) Provisional application No. 63/613,077, filed on Dec. 21, 2023.

(51) Int. Cl. B01D 53/14; B01J 8/02; C01B 17/04; C07C 7/11

(52) U.S. Cl. CPC B01D 53/1493; B01D 53/1425; B01D 53/1462; B01J 8/0278; C01B 17/0426; C07C 7/11; B01D 2252/20431; B01D 2252/20447; B01D 2252/20489; B01D 2256/245; B01D 2257/304; B01D 2257/504

(57) ABSTRACT

A process and gas processing plant configured to contact a produced gas stream with a lean aqueous solution comprising i) water and ii) an amine diol comprising a trisubstituted amine diol, a heterocyclic amine diol, or a combination of trisubstituted amine diol and a heterocyclic amine diol, to produce a treated gas stream comprising methane and a rich aqueous solution comprising the one or more impurities, the amine diol, and the water.

### CROSS-REFERENCE TO RELATED APPLICATION

This application is a non-provisional patent application claiming the benefit of, and priority to, U.S. Provisional Patent Application No. 63/613,077, filed Dec. 21, 2023, which is incorporated by reference herein in its entirety.

### FIELD OF THE DISCLOSURE

The present disclosure generally relates to the removal of impurities with chemical absorption compounds from a hydrocarbon-containing stream.

### BACKGROUND

Techniques for the capture of acid gases from gas streams can employ chemical solvents, such as methyldiethanolamine (MDEA), monoethanolamine (MEA), or diethanolamine (DEA), that can absorb one or more acid gases that contact the solvent. In practice, the solvent can be circulated through an absorption system having an absorber and regenerator. The solvent can feed to the absorber as a lean solvent. A feed gas stream containing one or more acid gases can be contacted with the lean liquid solvent in the absorber to absorb one or more acid gases into the liquid solvent. The liquid solvent loaded with acid gas(es) leaves the absorber as a rich solvent, and a treated gas stream having reduced amounts of acid gases leaves the absorber as well. The acid gases in the rich liquid solvent can be removed by techniques of solvent regeneration in the regenerator requiring the addition of heat to form the lean liquid solvent, which is recirculated to the absorber for additional acid gas capture.

Use of MDEA, MEA, or DEA often involves a high solvent circulation rate in the absorption system to achieve suitable levels of acid gas capture, which results in large amounts of the solvent used and large absorption and regeneration equipment, including equipment such as pumps and compressors. Pumps and compressors can be operated using an electric motor with electric power generated by an internal combustion engine. Therefore, some CO₂ emissions are created by the absorption system and are not absorbed by the solvent; thus, there is need to reduce CO₂ emissions from the absorption system.

### SUMMARY

Disclosed is a process that includes contacting a produced gas stream with a lean aqueous solution including i) water and ii) an amine diol including a trisubstituted amine diol, a heterocyclic amine diol, or a combination of the trisubstituted amine diol and the heterocyclic amine diol, to produce a treated gas stream including methane and a rich aqueous solution including the one or more impurities, the amine diol, and the water.

Also disclosed is a gas processing plant having an absorber configured to receive a produced gas stream and to contact a produced gas received from the produced gas stream with a lean aqueous solution including i) water and 2) an amine diol including a trisubstituted amine diol, a heterocyclic amine diol, or a combination of trisubstituted amine diol and a heterocyclic amine diol, to form a treated gas stream and a rich aqueous solution stream; and a regenerator having a liquid outlet coupled to a second inlet of the absorber and an inlet coupled to a liquid outlet of the absorber, wherein the regenerator is configured to receive the rich aqueous solution stream from the absorber and to strip impurities from the rich aqueous solution stream to form an impurity stream and a lean aqueous solution stream containing the lean aqueous solution.

Also disclosed is an aqueous solution comprising i) water and ii) an amine diol comprising a trisubstituted amine diol, a heterocyclic amine diol, or a combination of the trisubstituted amine diol and the heterocyclic amine diol.

Other technical features may be readily apparent to one skilled in the art from the following figures, descriptions, and claims.

### BRIEF DESCRIPTION OF THE DRAWINGS

For a more complete understanding of this disclosure, reference is now made to the following description, taken in conjunction with the accompanying drawings, in which:

FIG. 1 illustrates a side cross-sectional view of a wellbore environment in which the disclosed process and gas processing plant can be used.

FIG. 2 illustrates a schematic diagram of an embodiment of the disclosed process.

FIG. 3 illustrates a schematic diagram of an alternative embodiment of the disclosed process.

### DETAILED DESCRIPTION

The term “acid gas” as used herein refers to carbon dioxide (CO₂) and hydrogen sulfide (H₂S).

The term “sulfur-containing compound” as used herein refers to a molecule having one or more sulfur atoms in the chemical structure. Examples of sulfur-containing compounds as defined herein include carbonyl sulfide (COS) and compounds having the formula R-SH, where R is an alkyl group (e.g., methyl mercaptan, ethyl mercaptan, and so on) or an alcohol group (e.g., mercaptomethanol, 2-mercaptoethanol, 3-mercapto-1-propanol, and so on).

The terms “impurity” or “impurities” as used herein refer to any one or combination of the acid gases and the sulfur-containing compounds disclosed herein.

The term “light hydrocarbon gas” as used herein refers to saturated and unsaturated hydrocarbons that are in gas phase in a produced gas stream disclosed herein. For example, light hydrocarbon gases can have from 1 to 4 carbon atoms, such as methane, ethane, ethylene, propane, propylene, n-butane, i-butane, butylene, or combinations thereof.

“Condensates” as used herein refers to hydrocarbons produced in a well stream that have 5 to 12 carbon atoms, including linear or branched alkanes (e.g., pentane, hexane, heptane, and larger alkanes), cyclic alkanes (e.g., cyclohexane), linear or branched alkenes (e.g., pentene, hexene), aromatic compounds (benzene, toluene, xylenes, or combinations thereof), or combinations thereof.

“Wellbore” as used herein refers to a hole formed in a subterranean formation, for example, through drilling. The wellbore can be conventional (vertically oriented) or unconventional (having horizontally oriented portions). The wellbore can have a depth under the surface of up to about 10,000 ft, for example. Horizontal portions of an unconventional wellbore can extend laterally from a vertical portion and through a subterranean formation for a lateral distance that is up to about 10,000 ft. In aspects, a horizontal portion of the wellbore can have 30 to 90 stages.

“Crude oil” as used herein refers to hydrocarbons having 13 or more carbon atoms.

In the disclosed impurity capture processes and gas processing plant, it has been found that utilizing an aqueous solution of i) water and ii) an amine diol disclosed herein, to absorb impurities from a produced gas stream can result in a reduced solvent circulation rate, which reduces the size of equipment and energy needed for operation of the impurity capture processes and gas processing plant compared to traditional absorption solvents such as methyldiethanolamine (MDEA), monoethanolamine (MEA), or diethanolamine (DEA). The reduction in equipment size and operation energy reduces the carbon footprint of the disclosed impurity capture processes and gas processing plant compared to those impurity capture processes and gas processing plant that use MDEA, MEA, or DEA.

The amine diol disclosed herein can be one or more trisubstituted amine diol(s), one or more heterocyclic amine diol(s), or a combination of one or more trisubstituted amine diol(s) and one or more heterocyclic amine diol(s). An example of the amine diol is 3-pyrrolidino-1,2-propanediol. In aspects, the amine diol disclosed herein is the only amine-based absorption compound in the lean aqueous solution.

In aspects, the amine diol can be:

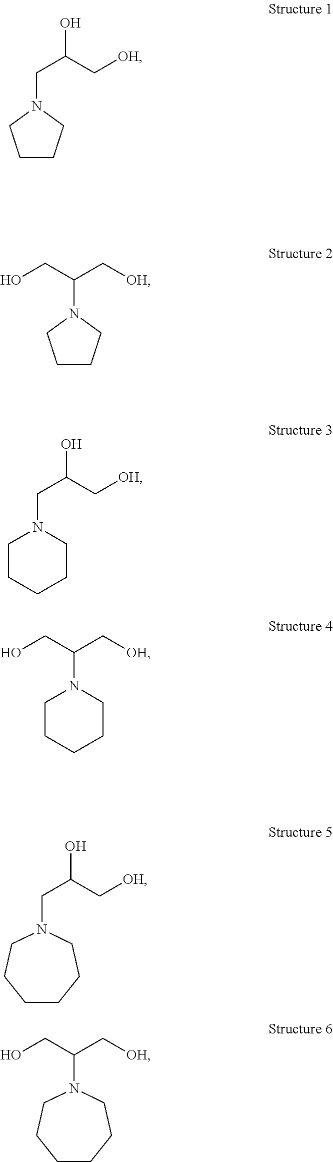

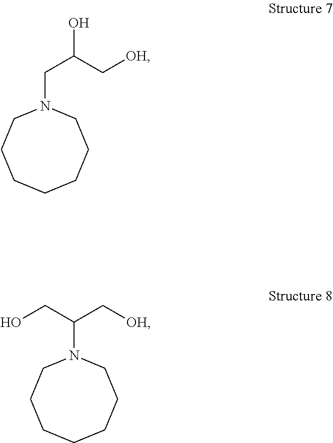 [SMILES: OCC(O)CN1CCCCCCC1 ; OCC(CO)N1CCCCCCC1]

or combinations thereof.

In aspects, the amine diol can be:

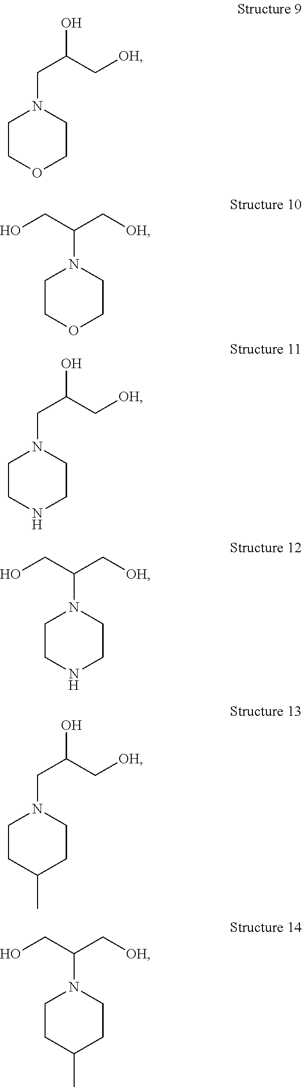

or combinations thereof.

In aspects, the amine diol can be:

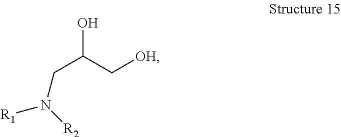

where R₁ is selected from hydrogen, a methyl group, an ethyl group, a propyl group, a butyl group, a pentyl group, a cyclopentyl group, a hexyl group, or a cyclohexyl group; and where R₂ is selected from hydrogen, a methyl group, an ethyl group, a propyl group, a butyl group, a pentyl group, a cyclopentyl group, a hexyl group, or a cyclohexyl group.

In aspects, the amine diol can comprise one or a combination of Structures 1 to 15 disclosed herein. In aspects, the amine diol which is embodied as one or a combination of Structures 1 to 15 is/are the only amine-based absorption compound(s) in the lean aqueous solution.

In aspects, the lean aqueous solution does not contain MDEA, MEA, or DEA, the rich aqueous solution does not contain MDEA, MEA, or DEA, or both the lean aqueous solution and the rich aqueous solution do not contain MDEA, MEA, or DEA.

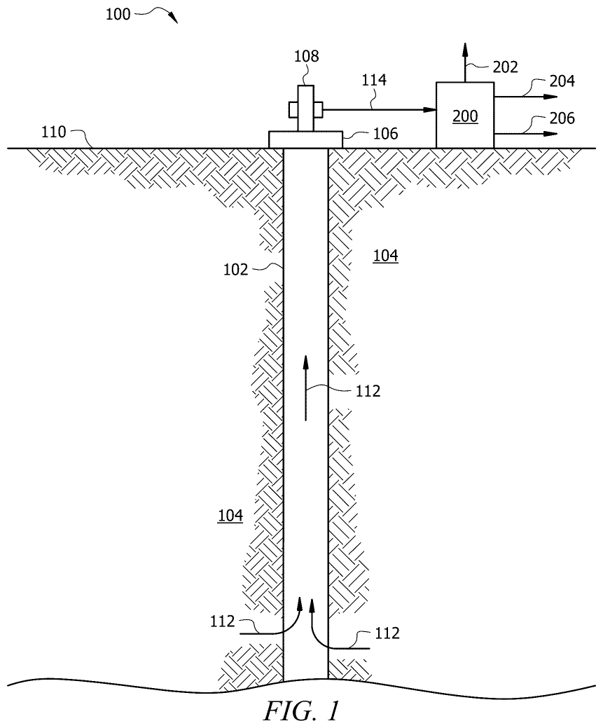

FIG. 1 illustrates a wellbore environment 100 in which the disclosed process and gas processing plant 200 can be used. The wellbore environment 100 includes a wellbore 102 formed in a subterranean formation 104. A well head 106 can cap the wellbore 102 at the surface 110 of the well site. A valve tree 108 (e.g., in embodiments, referred to as a Christmas tree) can be mechanically and fluidly connected to the well head 106 such that produced fluid 112 can be allowed to flow upward through the wellbore 102 to the surface 110, through the well head 106, and to the valve tree 108 (e.g., embodied as a suitable production valve tree). A well stream 114 can be fluidly connected to the valve tree 108 and to the gas processing plant 200 disclosed herein. The produced fluids 112 can flow through the valve tree 108, through well stream 114, and to the gas processing plant 200 of this disclosure. The well stream 114 can include, without limitation, crude oil, natural gases, condensates, water, proppant (e.g., sand), treatment additives (e.g., chemicals injected into the subterranean formation 104 to alter a chemistry within the formation to enhance hydrocarbon recovery), or combinations thereof. A pressure of the well stream 114 can be in a range of from about 2 MPaa to 100 MPaa, for example.

The gas processing plant 200 of the disclosure can be configured to separate the well stream 114 into one or more of a sales gas stream 202, a liquid stream 204, and an impurity stream 206. Additional detail about the gas processing plant 200 is discussed in the description below.

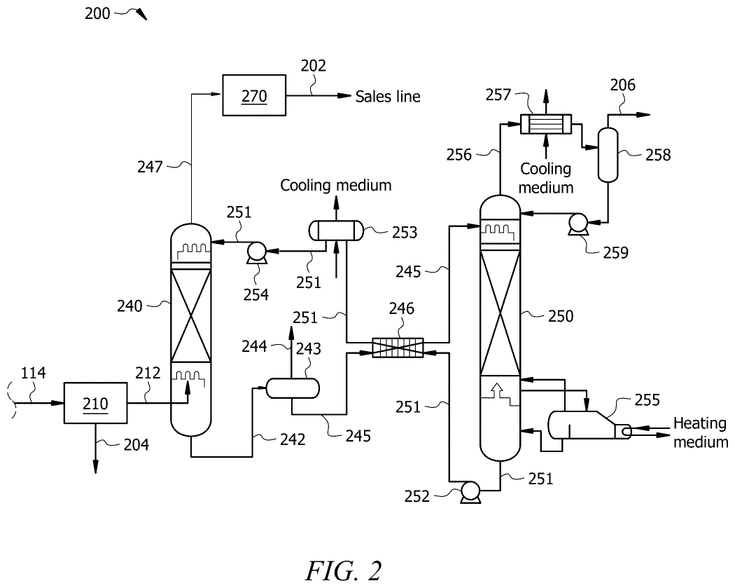

FIG. 2 illustrates a schematic diagram of an embodiment of the disclosed gas processing plant 200. While the gas processing plant 200 is described with reference to the equipment illustrated in FIG. 2, it should be appreciated that the action and functionality performed with the equipment illustrated in FIG. 2 can be performed in one or more aspects of a disclosed process.

The gas processing plant 200 is configured to receive the well stream 114 and to produce the sales gas stream 202, the liquid stream 204, and the impurity stream 206. The gas processing plant 200 can be configured to additionally produce a solids stream (e.g., containing reservoir solid particulates) and a C2+ stream 261 (e.g., containing ethane and heavier hydrocarbons) as described herein.

The gas processing plant 200 can include equipment 210 configured to recover the produced gas stream 212 that is fed to the absorber 240. The equipment 210 can include any separation equipment known in the art with the aid of this disclosure that is configured to recover the liquid stream 204 and the produced gas stream 202 from the well stream 114. For example, the equipment 210 can include one or more of a knockout pot (separation vessel to recover gas in one stream and liquid/solids in a second stream), a multiphase separator (separation vessel to recover gas, liquid, and solids in separate streams), a slug catcher, a plug catcher, a choke manifold, a distillation train, or combinations thereof.

The equipment 210 can include a slug catcher configured to receive the well stream 114 and to separate solid particulates (e.g., reservoir fines) from the well stream 114 to produce a first stream containing a wellbore fluid and a liquids stream containing accumulated periodic liquid slugs. A solids stream connected to slug catcher allows the periodic removal of accumulated separated reservoir fines from this vessel. The wellbore fluid 112 flows to the slug catcher via the well stream 114. Solid particulates removed by the slug catcher can include reservoir fines. Other solids, such as sand proppant, can also be removed by the slug catcher. An operating pressure of the slug catcher can be in a range of from about 2.0 MPaa to 100 MPaa, for example. The slug catcher can be embodied as a single vessel or multiple vessels in series, in parallel, or both in series and parallel, for example. The slug catcher can include one or more valves connected for intermittent or continuous removal of the solid particulates from the vessel(s) of the slug catcher, into the solids stream. The solid particulates in solids stream can be discharged into a tank, ditch, or onto the ground next to the slug catcher, for example.

The first stream produced by the slug catcher can include crude oil, natural gases, condensates, water, treatment additives (e.g., chemicals injected into the subterranean formation 104 to alter a chemistry within the formation to enhance hydrocarbon recovery), or combinations thereof. In aspects, the pressure of the first stream can be in a range of from about 2.0 MPaa to 100 MPaa, for example.

The equipment 210 can further include a choke manifold fluidly connected to the first stream. The choke manifold can be configured to receive the first stream from the slug catcher and to control a flow of fluids received from the first stream to produce a second stream having a flow rate suitable for introducing the second stream to a separator. The choke manifold can be a manifold assembly that can incorporate chokes, valves, and pressure sensors to provide controlled flow of fluids in second stream that are received by the choke manifold from first stream. The choke manifold can include flanged or integrated gate valves, positive chokes, and adjustable chokes, for example.

The second stream can include the same components of the first stream, e.g., crude oil, natural gases, condensates, water, proppant (e.g., sand), treatment additives (e.g., chemicals injected into the subterranean formation 104 to alter a chemistry within the formation to enhance hydrocarbon recovery), or combinations thereof.

The equipment 210 can further include a separator embodied as any vessel alone or in combination with other vessels and/or equipment that is configured to separate produced gases from the liquids and solids contained in the second stream, forming a gas stream (e.g., in some aspects, forming the produced gas stream 212) and the liquid stream 204. For example, when the second stream is embodied as a gas stream containing less than 50 wt % liquid phase components based on a total weight of the second stream, the separator of the equipment 210 can include a liquid knockout drum designed to separate the liquid and any solids from the gases and produce the gases in a gas stream (e.g., in some aspects, in the produced gas stream 212) and the liquids/solids in the liquids/solid stream 204. In another example, when the second stream is embodied as a liquid stream containing associated gases (e.g., less than 50 wt % gas phase components based on a total weight of the second stream), the separator can be embodied as a gas knockout drum designed to recover the associated gases in a gas stream (e.g., can be the produced gas stream 212) and liquid in the liquid stream 204.

The equipment 210 can further include a distillation train. The gases recovered from the above-described separator via a gas stream produced by the separator can flow to a distillation train containing a demethanizer and, in some aspects, one or more of a deethanizer and depropanizer, depending on the desired purity of ethane and propane that are recovered by the distillation train.

In aspects where the gas processing equipment 210 has a distillation train, the distillation train can be configured to receive the gas stream from the separator. The demethanizer (and any deethanizer and/or depropanizer) of the distillation train can include one or more distillation columns having trays and associated equipment (e.g., JT valve(s), condensers, reboiler, reflux separator, or combinations thereof) capable of recovering methane in an overhead stream of the demethanizer, forming the produced gas stream 212. C2+ hydrocarbons can flow in a bottoms stream from the demethanizer for further use or processing. In further processing, the bottoms stream of the demethanizer can flow to a deethanizer than can recover ethane in an overhead stream and C3+ hydrocarbons in a bottoms stream; alternatively, the bottoms stream of the demethanizer can flow to a depropanizer that can recover ethane and propane in an overhead stream and C4+ hydrocarbon in a bottoms stream; alternatively, the bottoms stream of the demethanizer can flow to a deethanizer than can recover ethane in an overhead stream and C3+ hydrocarbons in a bottoms stream, and the bottoms stream of the deethanizer can flow to a depropanizer that can recover ethane in an overhead stream and C4+ hydrocarbon in a bottoms stream. The distillation train can also include a cold box, heat exchanger, compressor, expander, or combinations thereof, as is known in the art for gas processing to recover methane from a stream containing light hydrocarbons.

In aspects, the produced gas stream 212 contains one or more light hydrocarbon gases and one or more impurities.

The gas processing plant 200 can further include an absorber 240 and a regenerator 250.

The produced gas stream 212 is configured to fluidly connect to an inlet of the absorber 240. The absorber 240 can include one or more vessels (also referred to as columns) and associated equipment (e.g., flash vessel 243, cooler 253, pump 254, trays, packing, valves, or combinations thereof) configured with operating conditions (e.g., temperature pressure, aqueous solution flow rate, or combinations thereof) to contact produced gas received from the produced gas stream 212 with a lean aqueous solution inside the absorber 240 to produce a treated gas that is recovered in treated gas stream 247 and a rich aqueous solution that is recovered in rich aqueous solution stream 242. Produced gas is received from the produced gas stream 212 and the lean aqueous solution is received from the lean aqueous solution stream 251. A flow of produced gas inside the absorber 240 is counter current with respect to a flow of the aqueous solution received from the lean aqueous solution stream 251 in the absorber 240. The aqueous solution flows from the inlet where the lean aqueous solution stream 251 connects to the absorber 240 (e.g., near the top of the absorber 240) in a direction toward the bottom of the absorber 240. The aqueous solution absorbs impurities (e.g., acid gases, sulfur-containing compounds, or a combination thereof) as the aqueous solution moves toward the bottom of the absorber 240, and the aqueous solution (also referred to as a loaded aqueous solution or rich aqueous solution, e.g., containing absorbed impurities and, in some aspects, co-absorbed hydrocarbons) then flows from the absorber 240 as a rich aqueous solution in the rich aqueous solution stream 242 that is connected to an outlet on the bottom of the absorber 240. Treated gas stream 247 contains the treated gas that flows from the top of absorber 240 having a lower concentration of impurities than a concentration of impurities in the produced gas stream 212.

The absorber 240 can operate at a temperature in a range of 25° C. to 70° C. and a pressure in a range of 0.2 MPaa to 10 MPaa. In aspects, a circulation rate of aqueous solution through the absorber 240 can be in a range of 100 to 3,000 liters/min. In some aspects, the circulation rate can be greater than 3,000 liters/min.

The produced gas in the produced gas stream 212 can include light hydrocarbon gases in a range of 60 vol % to 99 vol % and impurities in a range of 1 vol % to 20 vol % based on a total volume of the produced gas stream 212. In aspects, the impurities include both carbon dioxide in a range of 1 vol % to 20 vol % and hydrogen sulfide in a range of 0.0001 vol % to 10 vol % based on a total volume of the produced gas stream 212. In aspects, the produced gas in the produced gas stream 212 has no C5+ hydrocarbons (pentane or heavier hydrocarbons) or alternatively, can include C5+ hydrocarbons in a range of greater than 0 vol % and less than 3 vol % based on a total volume of the produced gas stream 212. In aspects, the pressure of the produced gas stream 212 can be in a range of 0.1 MPaa to about 8.0 MPaa.

The treated gas in the treated gas stream 247 can include light hydrocarbon gases in a range of 70 vol % to 100 vol % and impurities in a range of 0.00 vol % to 4.0 vol % based on a total volume of the treated gas stream 247. In aspects, the impurities include both carbon dioxide in a range of 0.00 vol % to 4.00 vol % and hydrogen sulfide in a range of 0.0001 vol % to 0.0004 vol % based on a total volume of the treated gas stream 247.

The lean aqueous solution stream 251 can include water in a range of 50 wt % to 85 wt %, the amine diol in a range of 15 wt % to 50 wt %, and impurities in a range of 0.10 wt % to 0.30 wt % based on a total weight of the lean aqueous solution stream 251. In aspects, the impurities include both carbon dioxide in a range of 0.05 wt % to 0.2 wt % and hydrogen sulfide in a range of 0.01 wt % to 0.1 wt % based on a total weight of the lean aqueous solution stream 251. The temperature of the lean aqueous solution stream 251 after cooling in the cooler 253 can be in a range of 35° C. to 60° C., for example, 45° C.

The rich aqueous solution stream 242 can include water in a range of 50 wt % to 85 wt %, the amine diol in a range of 15 wt % to 50 wt %, and impurities in a range of 1.0 wt % to 10.0 wt % based on a total weight of the rich aqueous solution stream 242. In some aspects, the rich aqueous solution stream 242 can additionally include light hydrocarbon gases in a range of 0.50 wt % to 2.0 wt % based on a total weight of the rich aqueous solution stream 242. In aspects, the impurities include both carbon dioxide in a range of 2.0 wt % to 4.0 wt % and hydrogen sulfide in a range of 1.0 wt % to 3.0 wt % based on a total weight of the rich aqueous solution stream 242.

In aspects, the loaded aqueous solution in the rich aqueous solution stream 242 can optionally flow to a flash vessel 243 (e.g., via a pressure reduction valve), where any co-absorbed volatile hydrocarbons and a portion of the absorbed impurities can flash from the rich aqueous solution. The flashed hydrocarbons and impurities can flow in gas phase from the flash vessel 243 into a vapor discharge stream 244, and the remaining rich aqueous solution flows in liquid phase from the flash vessel 243 via the second rich aqueous solution stream 245.

The second rich aqueous solution stream 245 can include water in a range of 50 wt % to 85 wt %, the amine diol in a range of 15 wt % to 50 wt %, and impurities in a range of 1.0 wt % to 10.0 wt % based on a total weight of the second rich aqueous solution stream 245. In some aspects, the second rich aqueous solution stream 245 can additionally include light hydrocarbon gases in a range of 0.01 wt % to 0.05 wt % based on a total weight of the second rich aqueous solution stream 245. In aspects, the impurities include both carbon dioxide in a range of 2.0 wt % to 4.0 wt % and hydrogen sulfide in a range of 1.0 wt % to 3.0 wt % based on a total weight of the second rich aqueous solution stream 245.

The second rich aqueous solution stream 245 can flow from the flash vessel 243 and through a cross-heat exchanger 246 where heat from the lean aqueous solution stream 251 is used to heat the second rich aqueous solution stream 245 to a temperature in a range of from 90° C. to 110° C., prior to introduction of the second rich aqueous solution stream 245 to the regenerator 250.

The second rich aqueous solution stream 245 is configured to fluidly connect to an inlet of the regenerator 250. The regenerator 250 can include one or more vessels (also referred to as columns) and associated equipment (e.g., condenser 257, reflux separator 258, pump 259, reboiler 255, trays, packing, valves, piping or combinations thereof) configured with operating conditions (temperature, pressure, aqueous solution circulation rate, or combinations thereof) to separate the impurities from the aqueous solution to produce the lean aqueous solution stream 251 containing the aqueous solution and a recovered gas stream 256 containing impurities that are liberated from the aqueous solution.

The regenerator 250 can operate at a temperature in a range of 100° C. to 135° C. and a pressure in a range of 0.2 MPaa to 0.4 MPaa. In aspects, a circulation rate of aqueous solution through the regenerator 250 can be in a range of 100 liters/min to 3,000 liters/min. In aspects, the circulation rate can be greater than 3,000 liters/min.

The impurities separate from the aqueous solution as the aqueous solution flows in a direction from the inlet to the bottom of the regenerator 250. In aspects, impurities can be stripped from the aqueous solution by a countercurrent flow of steam provided by the reboiler 255 to the bottom of the regenerator 250. The regenerated aqueous solution flows from the regenerator 250 in lean aqueous solution stream 251, and the impurities with steam flow from the regenerator 250 in recovered gas stream 256.

Steam and impurities flow through condenser 257, where steam condenses to water. Water and any other resulting liquids are separated from impurities in reflux separator 258. The water and any other liquids flow from the reflux separator 258 back to the top of the regenerator 250 via pump 259 (as a reflux for the regenerator 250). The impurities flow from the reflux separator 258 via impurity stream 206. The regenerated aqueous solution is pumped by pump 252 in the lean aqueous solution stream 251 for cooling in the cross-heat exchanger 246 and cooler 253, and for flow back into the top of absorber 240. Thus, a circuit or loop for the aqueous solution is made via lean aqueous solution stream 251, absorber 240, rich aqueous solution stream 242, rich aqueous solution stream 245, regenerator 250, and again to the lean aqueous solution stream 251.

In aspects, the impurity stream 206 includes hydrogen sulfide in a range of 0.01 vol % to 60 vol % and carbon dioxide in a range of 40 vol % to 99 vol % based on a total volume of the impurity stream 206. In additional aspects, light hydrocarbon gases may be present in a range of greater than 0 vol % to less than 5 vol % based on a total volume of the impurity stream 206.

The aqueous solution, on an acid-gas-free basis, comprises, consists of, or consists essentially of i) water and ii) an amine diol comprising a trisubstituted amine diol, a heterocyclic amine diol, or a combination of trisubstituted amine diol and a heterocyclic amine diol. Water is present in the aqueous solution in a range of 50 wt % to 99 wt %, alternatively, 60 wt % to 90 wt %, alternatively, 60 wt % to 80 t %, based on a total weight of the aqueous solution. The amine diol is present in the aqueous solution in a range of 1 wt % to 50 wt %, alternatively, 10 wt % to 50 wt %, alternatively, 20 wt % to 40 wt %, alternatively, 25 wt % to 40 wt %, based on a total weight of the aqueous solution.

In some aspects, the aqueous solution can additionally include one or more unsubstituted piperazine or substituted piperazine (e.g., substituted at any of the 1, 2, 3, 4, 5, or 6 position, or any combination of the 1, 2, 3, 4, 5, and 6 positions). When one or more piperazines are present in the aqueous solution, the concentration of all piperazine compound(s) in the aqueous solution, on an acid-gas-free basis, can be in a range of 1.0 wt % to 10.0 wt % based on a total weight of the aqueous solution.

Examples of mono-substituted piperazines include 2-aminopropyl-piperazine, and 2-aminobutyl-piperazine. Examples of bi-substituted piperazines include 1,4-bis-(2-aminopropyl)-piperazine; 1,4-bis-(2-aminobutyl)-piperazine; 1,4-bis-(3-aminobutyl)-piperazine; 1,4-bis-(N-methyl-aminoethyl)-piperazine; 1-(2-aminobutyl)-4-methylpiperazine; 1-(2-aminopropyl)-4-methylpiperazine; and 1-(2-aminopropyl)-4-ethylpiperazine; 1-aminoethyl-4-(2-aminobutyl)-piperazine; 1-aminoethyl-4-(2-aminopropyl)-piperazine; 1-aminopropyl-4-(3-aminobutyl)-piperazine; 1-aminoethyl-4-(N-methyl-aminoethyl)-piperazine; or combinations thereof.

Additional examples of piperazines include aminoethyl-piperazine; 2-aminoethyl-piperazine; 2-aminopropyl-piperazine; 2-aminobutyl-piperazine; 1-acetylpiperazine; 1-formylpiperazine; 1,4-bis-aminoethyl-piperazine; 1,4-bis-aminopropyl-piperazine; 1,4-bisaminobutyl-piper azine; 1,4-bis-(2-aminopropyl)-piperazine; 1,4-bis-(2-aminobutyl)-piperazine; 1,4-bis-(N-methyl-aminoethyl)-piperazine; 1-(2-aminobutyl)-4-methylpiperazine; 1-(2-aminopropyl)-4-methylpiperazine; 1-(2-aminopropyl)-4-ethylpiperazine; 1-aminoethyl-4-(2-aminobutyl)-piperazine; 1-aminoethyl-4-(2-aminopropyl)-piperazine; 1-aminoethyl-4-(N-methyl-aminoethyl)-piperazine; or combinations thereof.

The treated gas in treated gas stream 247 can flow to a sales pipeline as sales gas stream 202 or for further processing in gas processing equipment 270. FIG. 2 illustrates further processing in which the gas processing plant 200 further includes gas processing equipment 270 that produces the sales gas stream 202. In some aspects, the gas processing equipment 270 can include one or a series (stages) of gas compressors. In these aspects, a distillation train containing a demethanizer and one or more of a deethanizer and depropanizer can be included in the equipment 210. In additional or alternative aspects, the gas processing equipment 270 that is downstream of the absorber 240 can include a distillation train that has a demethanizer and, in some aspects, one or more of a deethanizer and depropanizer, depending on the desired purity of ethane and propane that are recovered by the distillation train.

In aspects where the gas processing equipment 270 has a distillation train, the distillation train can be configured to receive the treated gas stream 247. The demethanizer (and any deethanizer and/or depropanizer) of the distillation train can include one or more distillation columns having trays and associated equipment (e.g., JT valve(s), condensers, reboiler, reflux separator, or combinations thereof) capable of recovering methane in an overhead stream, that, if needed, can be compressed by one or more compressors as described above to form sales gas stream 202. C2+ hydrocarbons can flow in a bottoms stream from the demethanizer for further use or processing. In further processing, the bottoms stream can flow to a deethanizer than can recover ethane in an overhead stream and C3+ hydrocarbons in a bottoms stream; alternatively, the bottoms stream of the demethanizer can flow to a depropanizer that can recover ethane and propane in an overhead stream and C4+ hydrocarbon in a bottoms stream; alternatively, the bottoms stream of the demethanizer can flow to a deethanizer than can recover ethane in an overhead stream and C3+ hydrocarbons in a bottoms stream, and the bottoms stream of the deethanizer can flow to a depropanizer that can recover ethane in an overhead stream and C4+ hydrocarbon in a bottoms stream. The distillation train can also include a cold box, heat exchanger, compressor, expander, or combinations thereof, as is known in the art for gas processing to recover methane from a stream containing light hydrocarbons.

The sales gas stream 202 can flow to a sales gas line for transport or storage.

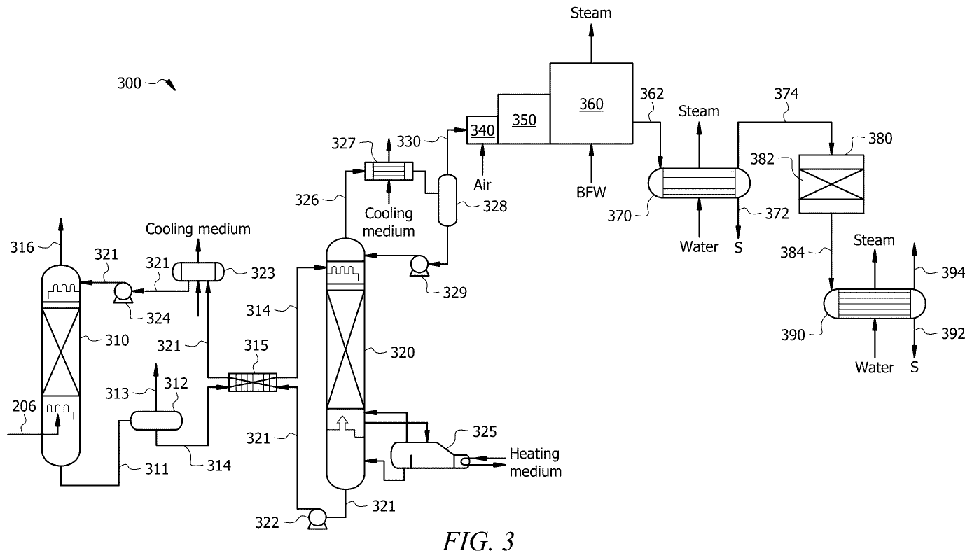

FIG. 3 illustrates a schematic diagram of an embodiment of the disclosed gas processing plant 300 that can be used in conjunction with the gas processing plant 200 of FIG. 2. While the gas processing plant 300 is described with reference to the equipment illustrated in FIG. 3, it should be appreciated that the action and functionality performed with the equipment illustrated in FIG. 3 can be performed in one or more aspects of a disclosed process.

The gas processing plant 300 is configured to receive the impurity stream 206 and to produce elemental sulfur in one or more elemental sulfur streams 372, 392. The gas processing plant 300 can include one or more of an absorber 310, a regenerator 320, a Claus burner 340, a first Claus reactor 350, a boiler 360, a condenser 370, a second Claus reactor 380, and a second condenser 390.

The impurity stream 206 is configured to fluidly connect to an inlet of the absorber 310. The absorber 310 can include one or more vessels (also referred to as columns) and associated equipment (e.g., flash vessel 312, cooler 323, pump 324, trays, packing, valves, or combinations thereof) configured with operating conditions (e.g., temperature pressure, aqueous solution flow rate, or combinations thereof) to contact one or more impurities received from the impurity stream 206 with a lean aqueous solution inside the absorber 310 to produce a treated gas that is recovered in treated gas stream 316 and a rich aqueous solution that is recovered in rich aqueous solution stream 311. Impurities are received from the impurity stream 206 and the lean aqueous solution is received from the lean aqueous solution stream 321. A flow of impurities inside the absorber 310 is counter current with respect to a flow of the aqueous solution received from the lean aqueous solution stream 321 in the absorber 310. The aqueous solution flows from the inlet where the lean aqueous solution stream 321 connects to the absorber 310 (e.g., near a top of the absorber 310) in a direction toward the bottom of the absorber 310. The aqueous solution absorbs impurities (e.g., one or more acid gases, one or more sulfur-containing compounds, or a combination thereof) as the aqueous solution moves toward the bottom of the absorber 310, and the aqueous solution (also referred to as a loaded aqueous solution or rich aqueous solution, e.g., containing absorbed impurities and, in some aspects, co-absorbed hydrocarbons) then flows from the absorber 310 as a rich aqueous solution in the rich aqueous solution stream 311 that is connected to an outlet on the bottom of the absorber 310. Treated gas stream 316 contains the treated gas that flows from the top of absorber 310 having a lower concentration of impurities than a concentration of impurities in the impurity stream 206.

The absorber 310 can operate at a temperature in a range of 25° C. to 70° C. and a pressure in a range of 0.2 MPaa to 0.4 MPaa. In aspects, a circulation rate of aqueous solution through the absorber 310 can be in a range of 100 liters/min to 2,000 liters/min.

In aspects, the impurity stream 206 has components in a concentration described hereinabove. The pressure of the impurity stream 206 can be in a range of from about 0.2 MPaa to about 0.4 MPaa.

The treated gas in the treated gas stream 316 can include light hydrocarbon gases in a range of 0.1 vol % to 5.0 vol % and impurities in a range of 95 vol % to 99.9 vol % based on a total volume of the treated gas stream 316. In aspects, the impurities include both carbon dioxide in a range of 95 vol % to 99.9 vol % and hydrogen sulfide in a range of 0.5 vol % to 5 vol % based on a total volume of the treated gas stream 316.

The lean aqueous solution stream 321 can include water in a range of 50 wt % to 85 wt %, the amine diol in a range of 15 wt % to 50 wt %, and impurities in a range of 0.1 wt % to 0.3 wt % based on a total weight of the lean aqueous solution stream 321. In aspects, the impurities include both carbon dioxide in a range of 0.05 wt % to 0.2 wt % and hydrogen sulfide in a range of 0.01 wt % to 0.1 wt % based on a total weight of the lean aqueous solution stream 321. The temperature of the lean aqueous solution stream 321 after cooling in the cross-heat exchanger 315 can be in a range of 35° C. to 60° C., for example, 45° C.

The rich aqueous solution stream 311 can include water in a range of 50 wt % to 85 wt %, the amine diol in a range of 15 wt % to 50 wt %, and impurities in a range of 1 wt % to 10 wt % based on a total weight of the rich aqueous solution stream 311. In some aspects, the rich aqueous solution stream 311 can additionally include light hydrocarbon gases in a range of 0.2 wt % to 2 wt % based on a total weight of the rich aqueous solution stream 311. In aspects, the impurities include both carbon dioxide in a range of 2 wt % to 4 wt % and hydrogen sulfide in a range of 1 wt % to 3 wt % based on a total weight of the rich aqueous solution stream 311.

In aspects, the loaded aqueous solution in the rich aqueous solution stream 311 can optionally flow to a flash vessel 312 (e.g., via a pressure reduction valve), where any co-absorbed volatile hydrocarbons and a portion of the absorbed impurities can flash from the rich aqueous solution. The flashed hydrocarbons and impurities can flow in gas phase from the flash vessel 312 into a vapor discharge stream 313, and the remaining rich aqueous solution flows in liquid phase from the flash vessel 312 via the second rich aqueous solution stream 314.

The second rich aqueous solution stream 314 can include water in a range of 50 wt % to 85 wt %, the amine diol in a range of 15 wt % to 50 wt %, and impurities in a range of 1 wt % to 10 wt % based on a total weight of the second rich aqueous solution stream 314. In some aspects, the second rich aqueous solution stream 314 can additionally include light hydrocarbon gases in a range of 0.01 wt % to 0.05 wt % based on a total weight of the second rich aqueous solution stream 314. In aspects, the impurities include both carbon dioxide in a range of 2 wt % to 4 wt % and hydrogen sulfide in a range of 1 wt % to 3 wt % based on a total weight of the second rich aqueous solution stream 314.

The second rich aqueous solution stream 314 can flow from the flash vessel 312 and through a cross-heat exchanger 315 where heat from the lean aqueous solution stream 321 is used to heat the second rich aqueous solution stream 314 to a temperature in a range of from 90° C. to 110° C., prior to introduction of the second rich aqueous solution stream 314 to the regenerator 320.

The second rich aqueous solution stream 314 is configured to fluidly connect to an inlet of the regenerator 320. The regenerator 320 can include one or more vessels (also referred to as columns) and associated equipment (e.g., condenser 327, reflux separator 328, pump 329, reboiler 325, trays, packing, valves, piping or combinations thereof) configured with operating conditions (temperature, pressure, aqueous solution circulation rate, or combinations thereof) to separate the impurities from the aqueous solution to produce the lean aqueous solution stream 321 containing the aqueous solution and a recovered gas stream 326 containing impurities that are liberated from the aqueous solution.

The regenerator 320 can operate at a temperature in a range of 100° C. to 135° C. and a pressure in a range of 0.2 MPaa to 0.4 MPaa. In aspects, a circulation rate of aqueous solution through the regenerator 320 can be in a range of 100 to 3,000 liters/min. In some aspects, the circulation rate can be greater than 3,000 liters/min.

The impurities separate from the aqueous solution as the aqueous solution flows in a direction from the inlet to the bottom of the regenerator 320. In aspects, impurities can be stripped from the aqueous solution by a countercurrent flow of steam provided by the reboiler 325 to the bottom of the regenerator 320. The regenerated aqueous solution flows from the regenerator 320 in lean aqueous solution stream 321, and the impurities with steam flow from the regenerator 320 in recovered gas stream 326.

Steam and impurities flow through condenser 327, where steam condenses to water. Water and any other resulting liquids are separated from impurities in reflux separator 328. The water and any other liquids flow from the reflux separator 258 back to the top of the regenerator 320 via pump 329 (as a reflux for the regenerator 320). The impurities flow from the reflux separator 328 via a second impurity stream 330. The regenerated aqueous solution is pumped by pump 322 in the lean aqueous solution stream 321 for cooling in the cross-heat exchanger 315 and cooler 323, and then for flow back into the top of absorber 310. Thus, a circuit or loop for the aqueous solution is made via lean aqueous solution stream 321, absorber 310, rich aqueous solution stream 311, rich aqueous solution stream 314, regenerator 320, and again to the lean aqueous solution stream 321.

In aspects, the second impurity stream 330 includes hydrogen sulfide in a range of 25 vol % to 65 vol % and carbon dioxide in a range of 35 vol % to 75 vol % based on a total volume of the second impurity stream 330. In additional aspects, light hydrocarbon gases may be present in a range of greater than 0 vol % to less than 5 vol % based on a total volume of the second impurity stream 330.

The second impurity stream 330 is then configured to connect to an inlet of a burner 340 of the Claus unit for the Claus process, where the impurities are combined with air and heated. The components are then fed to the first Claus reactor 350 for thermally driven combustion of hydrogen sulfide in the presence of oxygen to form elemental sulfur (solid particles) and water vapor. An exemplary temperature and pressure of the first Claus reactor 350 is a temperature greater than 1050° C. and a pressure of 0.25 MPaa. The fluidized product then flows into a boiler 360 where the heat of the fluidized product transfers to water to form steam, resulting in a cooling of the fluidized product. The cooled fluidized product then flossed in stream 362 to a first condenser, where water is again used to cool the fluidized product. Elemental sulfur can be recovered in stream 372 from the condenser, and separated gas still containing hydrogen sulfide flows from the condenser 370 in stream 374 to the second Claus reactor 380. The second Claus reactor 380 is configured for a catalyzed reaction of hydrogen sulfide to elemental sulfur, albeit at lower temperature than the first Claus reactor 350. The catalyst can be contained in a catalyst bed 382, and the catalyst can be activated alumina or activated titania, for example. An exemplary temperature for the second Claus reactor 380 is in a range of 200° C. to 305° C. The reaction product of the second Claus reactor 380 flows from the reactor 380 in stream 384 to a second condenser 390, where like the first condenser 370, water is used to cool the reaction product for recovery of elemental sulfur in stream 392 and recovered gases in stream 394. Subsequent catalyzed reactor/condenser gas processing plant can be used for further treatment of stream 394. In FIG. 3, stream 394 can be referred o as a Claus tail gas, which can be treated in a tail gas treatment unit according to techniques known in the art with the aid of this disclosure.

### ASPECTS

Aspect 1. A process comprising: contacting a produced gas stream with a lean aqueous solution comprising i) water and ii) an amine diol comprising a trisubstituted amine diol, a heterocyclic amine diol, or a combination of the trisubstituted amine diol and the heterocyclic amine diol, to produce a treated gas stream comprising methane and a rich aqueous solution comprising one or more impurities, the amine diol, and the water.

Aspect 2. The process of Aspect 1, wherein the amine diol comprises:

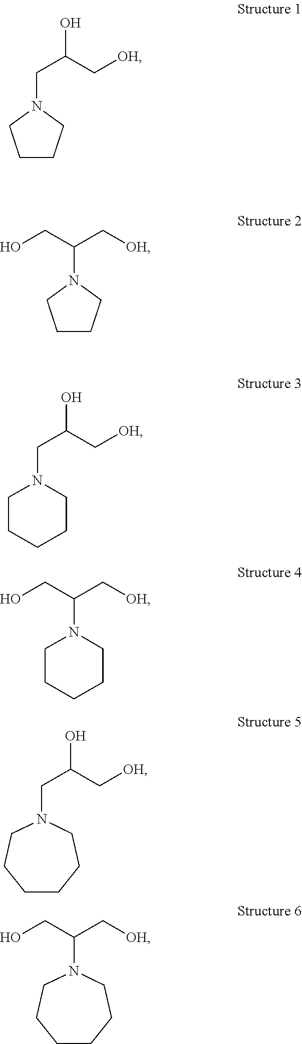

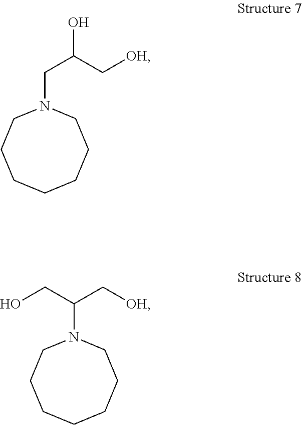 [SMILES: OCC(O)CN1CCCCCCC1 ; OCC(CO)N1CCCCCCC1]

or combinations thereof.

Aspect 3. The process of Aspect 1 or 2, wherein the amine diol comprises:

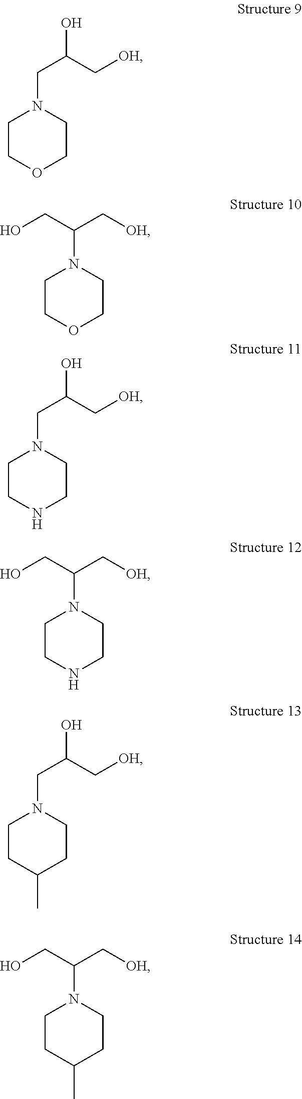

or combinations thereof.

Aspect 4. The process of any one of Aspects 1 to 3, wherein the amine diol comprises:

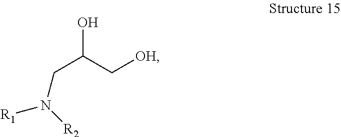

wherein R1 is selected from hydrogen, a methyl group, an ethyl group, a propyl group, a butyl group, a pentyl group, a cyclopentyl group, a hexyl group, or a cyclohexyl group; and wherein R2 is selected from hydrogen, a methyl group, an ethyl group, a propyl group, a butyl group, a pentyl group, a cyclopentyl group, a hexyl group, or a cyclohexyl group.

Aspect 5. The process of any one of Aspects 1 to 4, wherein the amine diol is the only amine-based absorption compound in the lean aqueous solution.

Aspect 6. The process of any one of Aspects 1 to 5, wherein the lean aqueous solution contains less than 50 wt % of the amine diol based on a total weight of the lean aqueous solution.

Aspect 7. The process of any one of Aspects 1 to 6, wherein the lean aqueous solution contains 15 wt % to 50 wt % of the amine diol based on a total weight of the lean aqueous solution.

Aspect 8. The process of any one of Aspects 1 to 7, wherein the lean aqueous solution further comprises a piperazine compound.

Aspect 9. The process of any one of Aspects 1 to 8, further comprising: producing a sales gas stream from the treated gas stream.

Aspect 10. The process of any one of Aspects 1 to 9, further comprising: stripping the one or more impurities with a stripping gas from the rich aqueous solution to form the lean aqueous solution and an impurity stream, wherein the stripping gas is steam.

Aspect 11. The process of Aspect 10, wherein the impurity stream is a first impurity stream, the process further comprising: contacting the first impurity stream with a second lean aqueous solution comprising water and the amine diol to produce a second treated gas stream comprising the methane and a second rich aqueous solution comprising the one or more impurities, the amine diol, and the water; and stripping the one or more impurities with a second stripping gas from the second rich aqueous solution to form a second lean aqueous solution and a second impurity stream, wherein the second stripping gas is steam, wherein the first impurity stream has a first concentration of hydrogen sulfide that is less than a second concentration of hydrogen sulfide in the second impurity stream.

Aspect 12. The process of Aspect 11, further comprising: subjecting the second impurity stream to a Claus process to form elemental sulfur.

Aspect 13. The process of any one of Aspects 1 to 12, further comprising: receiving a well stream from a wellbore; and separating the well stream into one or more streams comprising the produced gas stream, wherein the produced gas stream comprises one or more light hydrocarbon gases and the one or more impurities are selected from an acid gas, a sulfur-containing compound, or a combination thereof.

Aspect 14. The process of Aspect 13, wherein the one or more streams further comprises a liquid stream.

Aspect 15. The process of Aspect 13 or 14, wherein the one or more streams further comprises a solids stream.

Aspect 16. A gas processing plant comprising: an absorber configured to receive a produced gas stream comprising methane and one or more impurities and to contact a produced gas received from the produced gas stream with a lean aqueous solution comprising i) water and 2) an amine diol comprising a trisubstituted amine diol, a heterocyclic amine diol, or a combination of trisubstituted amine diol and a heterocyclic amine diol, to form a treated gas stream comprising the methane and a rich aqueous solution stream comprising the one or more impurities; and a regenerator having a liquid outlet coupled to a second inlet of the absorber and an inlet coupled to a liquid outlet of the absorber, wherein the regenerator is configured to receive the rich aqueous solution stream from the absorber and to strip impurities from the rich aqueous solution stream to form an impurity stream comprising the one or more impurities and a lean aqueous solution stream containing the lean aqueous solution.

Aspect 17. The gas processing plant of Aspect 16, wherein the amine diol comprises:

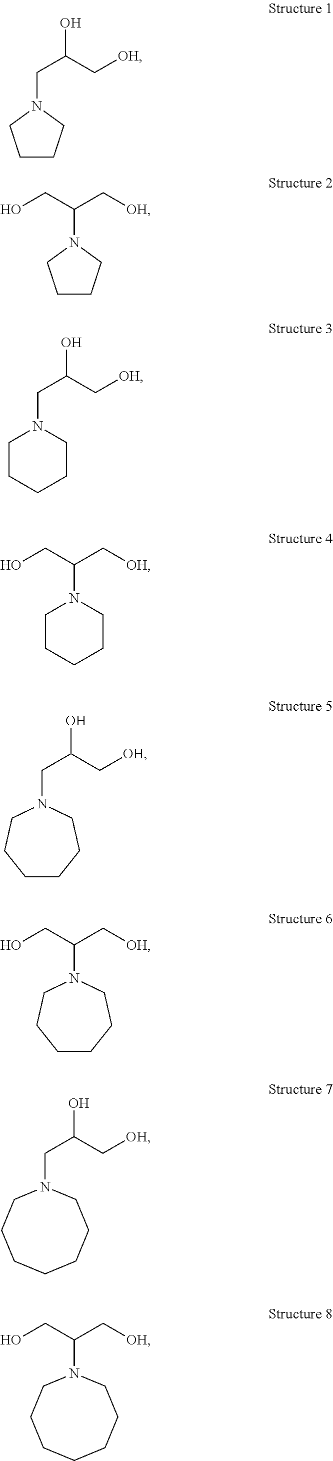

or combinations thereof.

Aspect 18. The gas processing plant of Aspect 16 or 17, wherein the amine diol comprises:

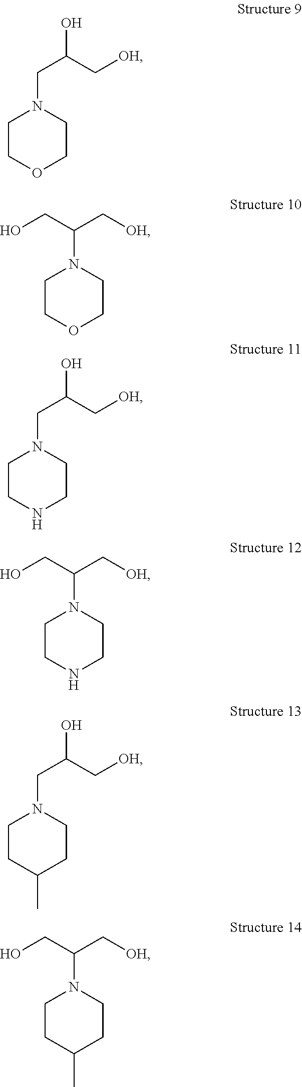

or combinations thereof.

Aspect 19. The gas processing plant of any one of Aspects 16 to 18, wherein the amine diol comprises:

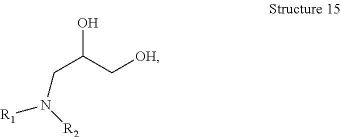

wherein R1 is selected from hydrogen, a methyl group, an ethyl group, a propyl group, a butyl group, a pentyl group, a cyclopentyl group, a hexyl group, or a cyclohexyl group; and wherein R2 is selected from hydrogen, a methyl group, an ethyl group, a propyl group, a butyl group, a pentyl group, a cyclopentyl group, a hexyl group, or a cyclohexyl group.

Aspect 20. The gas processing plant of any one of Aspects 16 to 19, wherein the amine diol is the only amine-based absorption compound in the lean aqueous solution.

Aspect 21. The gas processing plant of any one of Aspects 16 to 20, wherein the lean aqueous solution contains less than 50 wt % of the amine diol on a total weight of the lean aqueous solution.

Aspect 22. The gas processing plant of any one of Aspects 16 to 21, wherein the lean aqueous solution contains 15 wt % to 40 wt % of the amine diol based on a total weight of the lean aqueous solution.

Aspect 23. The gas processing plant of any one of Aspects 16 to 22, wherein the lean aqueous solution further comprises a piperazine compound.

Aspect 24. The gas processing plant of any one of Aspects 16 to 23, further comprising: gas processing equipment having an inlet coupled to a gas outlet of the absorber, wherein the gas processing equipment is configured to produce a sales gas stream comprising methane from the treated gas stream.

Aspect 25. The gas processing plant of any one of Aspects 16 to 24, further comprising: a second absorber having a first inlet coupled to a gas outlet of the regenerator, wherein the second absorber is configured to receive the impurity stream comprising the one or more impurities and further configured to contact the one or more impurities received from the impurity stream with a second lean aqueous solution comprising i) water and ii) the amine diol, to form a second treated gas stream and a second rich aqueous solution stream; and a second regenerator having a liquid outlet coupled to a second inlet of the second absorber and an inlet coupled to a liquid outlet of the second absorber, wherein the second regenerator is configured to receive the second rich aqueous solution stream from the second absorber and to strip the one or more impurities from the second rich aqueous solution stream to form a second impurity stream comprising the one or more impurities and a second lean aqueous solution stream containing the second lean aqueous solution.

Aspect 26. The gas processing plant of Aspect 25, further comprising: a Claus unit comprising a burner, a thermal Claus reactor, a boiler, a first condenser, a catalytic Claus reactor, and a second condenser, wherein the Claus unit is configured to receive the second impurity stream and convert hydrogen sulfide received from the second impurity stream to elemental sulfur.

Aspect 27. The gas processing plant of any one of Aspects 16 to 26, wherein the one or more impurities comprises an acid gas, a sulfur-containing compound, or any combination thereof.

Aspect 28. The gas processing plant of Aspect 27, wherein the sulfur-containing compound comprises carbonyl sulfide, a thiol, or both.

Aspect 29. An aqueous solution comprising i) water and ii) an amine diol comprising a trisubstituted amine diol, a heterocyclic amine diol, or a combination of the trisubstituted amine diol and the heterocyclic amine diol.

Aspect 30. The aqueous solution of Aspect 29, wherein the amine diol comprises:

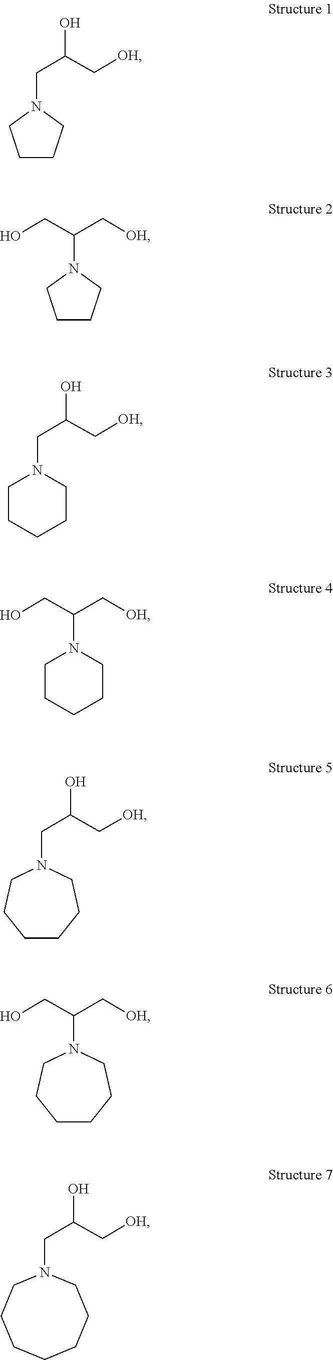

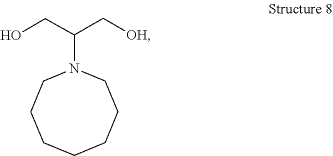 [SMILES: OCC(CO)N1CCCCCCC1]

or combinations thereof.

Aspect 31. The aqueous solution of Aspect 29 or 30, wherein the amine diol comprises:

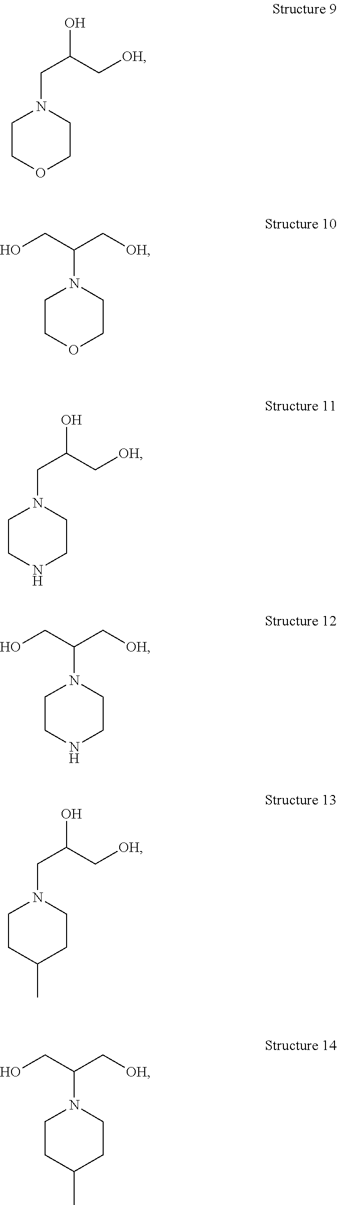

or combinations thereof.

Aspect 32. The aqueous solution of any one of Aspects 29 to 31, wherein the amine diol comprises:

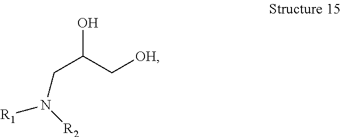

wherein R1 is selected from hydrogen, a methyl group, an ethyl group, a propyl group, a butyl group, a pentyl group, a cyclopentyl group, a hexyl group, or a cyclohexyl group; and wherein R2 is selected from hydrogen, a methyl group, an ethyl group, a propyl group, a butyl group, a pentyl group, a cyclopentyl group, a hexyl group, or a cyclohexyl group.

Aspect 33. The aqueous solution of any one of Aspects 29 to 32, wherein the amine diol is the only amine-based absorption compound in the aqueous solution.

Aspect 34. The aqueous solution of any one of Aspects 29 to 33, containing less than 50 wt % of the amine diol based on a total weight of the aqueous solution.

Aspect 35. The aqueous solution of any one of Aspects 29 to 34, containing from 15 wt % to 50 wt % of the amine diol based on a total weight of the aqueous solution.

Aspect 36. The aqueous solution of any one of Aspects 29 to 35, wherein the aqueous solution further comprises a piperazine compound.

Although the present disclosure and its advantages have been described in detail, it should be understood that various changes, substitutions, and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A process comprising:
contacting a produced gas stream with a lean aqueous solution comprising i) water and ii) an amine diol comprising a trisubstituted amine diol, a heterocyclic amine diol, or a combination of the trisubstituted amine diol and the heterocyclic amine diol, to produce a treated gas stream comprising methane and a rich aqueous solution comprising one or more impurities, the amine diol, and the water.

2. The process of claim 1, wherein the amine diol comprises: 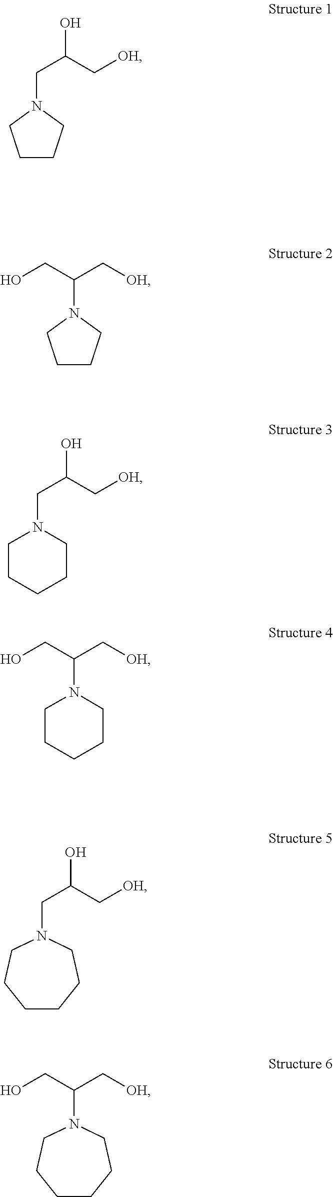 [SMILES: OCC(O)CN1CCCC1 ; OCC(CO)N1CCCC1 ; OCC(O)CN1CCCCC1 ; OCC(CO)N1CCCCC1 ; OCC(O)CN1CCCCCC1 ; OCC(CO)N1CCCCCC1] 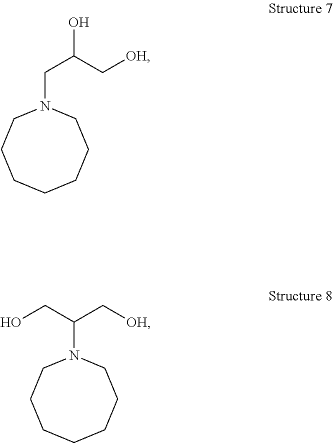 [SMILES: OCC(O)CN1CCCCCCC1 ; OCC(CO)N1CCCCCCC1]
or combinations thereof.

3. The process of claim 1, wherein the amine diol comprises: 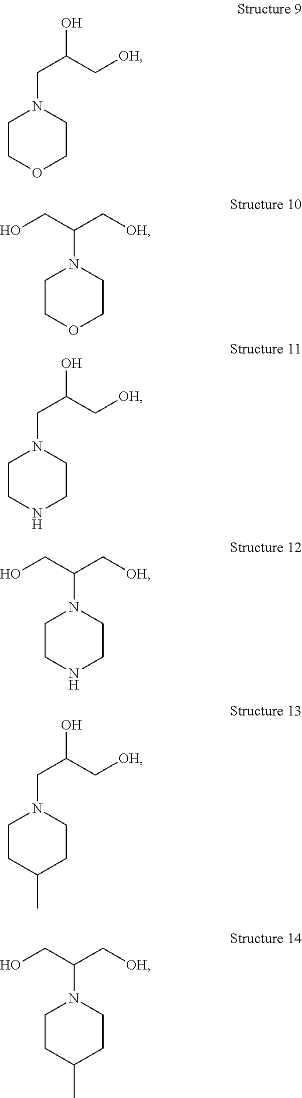
or combinations thereof.

4. The process of any claim 1, wherein the amine diol comprises: 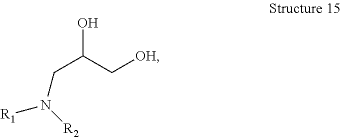
wherein R₁ is selected from hydrogen, a methyl group, an ethyl group, a propyl group, a butyl group, a pentyl group, a cyclopentyl group, a hexyl group, or a cyclohexyl group; and wherein R₂ is selected from hydrogen, a methyl group, an ethyl group, a propyl group, a butyl group, a pentyl group, a cyclopentyl group, a hexyl group, or a cyclohexyl group.

5. The process of claim 1, wherein the amine diol is the only amine-based absorption compound in the lean aqueous solution.

6. The process of claim 1, wherein the lean aqueous solution contains less than 50 wt % of the amine diol based on a total weight of the lean aqueous solution.

7. The process of claim 1, wherein the lean aqueous solution contains 15 wt % to 50 wt % of the amine diol based on a total weight of the lean aqueous solution.

8. The process of claim 1, wherein the lean aqueous solution further comprises a piperazine compound.

9. The process of claim 1, further comprising:
producing a sales gas stream from the treated gas stream.

10. The process of claim 1, further comprising:
stripping the one or more impurities with a stripping gas from the rich aqueous solution to form the lean aqueous solution and an impurity stream, wherein the stripping gas is steam.

11. The process of claim 10, wherein the impurity stream is a first impurity stream, the process further comprising:
contacting the first impurity stream with a second lean aqueous solution comprising water and the amine diol to produce a second treated gas stream comprising the methane and a second rich aqueous solution comprising the one or more impurities, the amine diol, and the water; and
stripping the one or more impurities with a second stripping gas from the second rich aqueous solution to form a second lean aqueous solution and a second impurity stream, wherein the second stripping gas is steam, wherein the first impurity stream has a first concentration of hydrogen sulfide that is less than a second concentration of hydrogen sulfide in the second impurity stream.

12. The process of claim 11, further comprising:
subjecting the second impurity stream to a Claus process to form elemental sulfur.

13. The process of claim 1, further comprising:
receiving a well stream from a wellbore; and
separating the well stream into one or more streams comprising the produced gas stream, wherein the produced gas stream comprises one or more light hydrocarbon gases and the one or more impurities are selected from an acid gas, a sulfur-containing compound, or a combination thereof.

14. The process of claim 13, wherein the one or more streams further comprises a liquid stream.

15. The process of claim 13, wherein the one or more streams further comprises a solids stream.

16. A gas processing plant comprising:
an absorber configured to receive a produced gas stream comprising methane and one or more impurities and to contact a produced gas received from the produced gas stream with a lean aqueous solution comprising i) water and 2) an amine diol comprising a trisubstituted amine diol, a heterocyclic amine diol, or a combination of trisubstituted amine diol and a heterocyclic amine diol, to form a treated gas stream comprising the methane and a rich aqueous solution stream comprising the one or more impurities; and
a regenerator having a liquid outlet coupled to a second inlet of the absorber and an inlet coupled to a liquid outlet of the absorber, wherein the regenerator is configured to receive the rich aqueous solution stream from the absorber and to strip impurities from the rich aqueous solution stream to form an impurity stream comprising the one or more impurities and a lean aqueous solution stream containing the lean aqueous solution.

17. The gas processing plant of claim 16, further comprising:
gas processing equipment having an inlet coupled to a gas outlet of the absorber, wherein the gas processing equipment is configured to produce a sales gas stream comprising methane from the treated gas stream.

18. The gas processing plant of claim 16, further comprising:
a second absorber having a first inlet coupled to a gas outlet of the regenerator, wherein the second absorber is configured to receive the impurity stream comprising the one or more impurities and further configured to contact the one or more impurities received from the impurity stream with a second lean aqueous solution comprising i) water and ii) the amine diol, to form a second treated gas stream and a second rich aqueous solution stream;
a second regenerator having a liquid outlet coupled to a second inlet of the second absorber and an inlet coupled to a liquid outlet of the second absorber, wherein the second regenerator is configured to receive the second rich aqueous solution stream from the second absorber and to strip the one or more impurities from the second rich aqueous solution stream to form a second impurity stream comprising the one or more impurities and a second lean aqueous solution stream containing the second lean aqueous solution; and
a Claus unit comprising a burner, a thermal Claus reactor, a boiler, a first condenser, a catalytic Claus reactor, and a second condenser, wherein the Claus unit is configured to receive the second impurity stream and convert hydrogen sulfide received from the second impurity stream to elemental sulfur.

19. An aqueous solution comprising i) water and ii) an amine diol comprising a trisubstituted amine diol, a heterocyclic amine diol, or a combination of the trisubstituted amine diol and the heterocyclic amine diol.

20. The aqueous solution of claim 19, wherein the amine diol comprises: 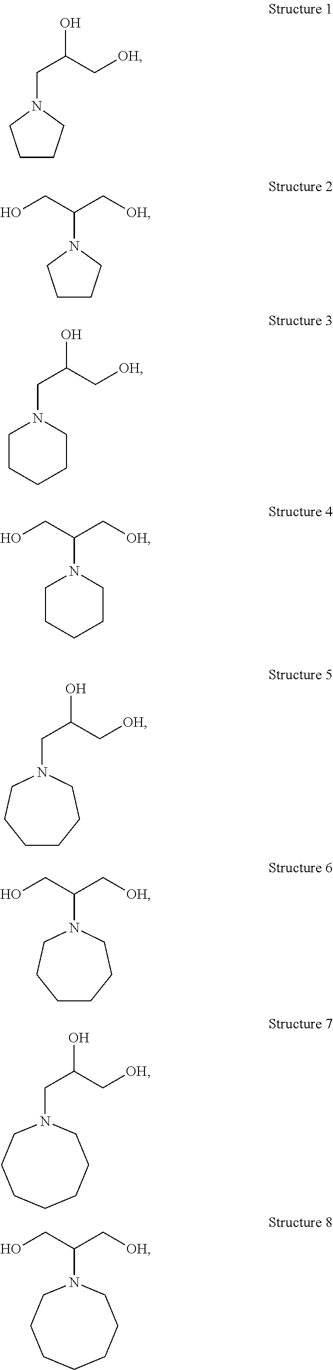 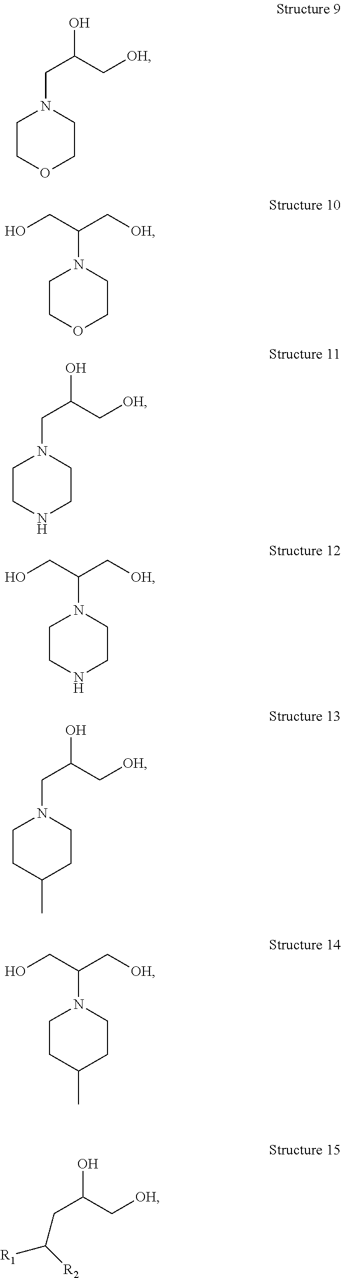
or combinations thereof, wherein R₁ is selected from hydrogen, a methyl group, an ethyl group, a propyl group, a butyl group, a pentyl group, a cyclopentyl group, a hexyl group, or a cyclohexyl group; and wherein R₂ is selected from hydrogen, a methyl group, an ethyl group, a propyl group, a butyl group, a pentyl group, a cyclopentyl group, a hexyl group, or a cyclohexyl group.
